(19) United States
(12) Patent Application Publication
(10) Pub. No.: US 2009/0062656 A1
(43) Pub. Date: Mar. 5, 2009

(54) BACKING MATERIAL, ULTRASONIC PROBE, ULTRASONIC ENDOSCOPE, ULTRASONIC DIAGNOSTIC APPARATUS, AND ULTRASONIC ENDOSCOPIC APPARATUS

(75) Inventor: Hiroaki HYUGA, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Tokyo (JP)

(21) Appl. No.: 12/171,667

(22) Filed: Jul. 11, 2008

(30) Foreign Application Priority Data
Sep. 3, 2007 (JP) 2007-227641

(51) Int. Cl. A61B 8/00

(52) U.S. Cl. 600/459

(57) ABSTRACT

A backing material for suppressing the surface temperature rise of an ultrasonic probe. This backing material is provided on a back face of at least one vibrator for transmitting and/or receiving ultrasonic waves in an ultrasonic probe, and includes: a backing base material containing a polymeric material; and a heat conducting fiber provided in the backing base material, having a larger coefficient of thermal conductivity than that of the backing base material, and running through without disconnection from a first face of the backing material in contact with the at least one vibrator to a second face different from the first face.

### BACKGROUND OF THE INVENTION

1. Field of the Invention

The present invention relates to a backing material to be used in an ultrasonic probe for transmitting and receiving ultrasonic waves. Further, the present invention relates to an ultrasonic probe to be used when intracavitary scan or extracavitary scan is performed on an object to be inspected, and an ultrasonic endoscope to be used by being inserted into a body cavity of the object, each including such a backing material. Furthermore, the present invention relates to an ultrasonic diagnostic apparatus or ultrasonic endoscopic apparatus including such an ultrasonic probe or ultrasonic endoscope and a main body unit.

2. Description of a Related Art

In medical fields, various imaging technologies have been developed in order to observe the interior of an object to be inspected and make diagnoses. Especially, ultrasonic imaging for acquiring interior information of the object by transmitting and receiving ultrasonic waves enables image observation in real time and provides no exposure to radiation unlike other medical image technologies such as X-ray photography or RI (radio isotope) scintillation camera. Accordingly, ultrasonic imaging is utilized as an imaging technology at a high level of safety in a wide range of departments including not only the fetal diagnosis in the obstetrics, but also gynecology, circulatory system, digestive system, and so on.

The ultrasonic imaging is an image generation technology utilizing the nature of ultrasonic waves that the waves are reflected at a boundary between regions with different acoustic impedances (e.g., a boundary between structures). Typically, an ultrasonic diagnostic apparatus (or referred to as an ultrasonic imaging apparatus or an ultrasonic observation apparatus) is provided with an ultrasonic probe to be used in contact with the object or ultrasonic probe to be used by being inserted into a body cavity of the object. Alternatively, an ultrasonic endoscope in combination of an endoscope for optically observing the interior of the object and an ultrasonic probe for intracavity is also used.

Using such an ultrasonic probe or ultrasonic endoscope, ultrasonic beams are transmitted toward the object such as a human body and ultrasonic echoes generated by the object are received, and thereby, ultrasonic image information is acquired. On the basis of the ultrasonic image information, ultrasonic images of structures (e.g., internal organs, diseased tissues, or the like) existing within the object are displayed on a display unit of the ultrasonic diagnostic apparatus.

In the ultrasonic probe, a vibrator (piezoelectric vibrator) having electrodes formed on both sides of a material that expresses piezoelectric effect (a piezoelectric material) is generally used as an ultrasonic transducer for transmitting and receiving ultrasonic waves. As the piezoelectric material, a piezoelectric ceramics represented by PZT (Pb(lead) zirconate titanate), a polymeric piezoelectric material represented by PVDF (polyvinylidene difluoride), or the like is used.

When a voltage is applied to the electrodes of the vibrator, the piezoelectric material expands and contracts due to the piezoelectric effect to generate ultrasonic waves. Accordingly, plural vibrators are one-dimensionally or two-dimensionally arranged and the vibrators are sequentially driven, and thereby, an ultrasonic beam transmitted in a desired direction can be formed. Further, the vibrator receives the propagating ultrasonic waves, expands and contracts, and generates an electric signal. The electric signal is used as a reception signal of ultrasonic waves.

When ultrasonic waves are transmitted, drive signals having great energy are supplied to the ultrasonic transducers. However, not the whole energy of the drive signals is converted into acoustic energy but the considerable amount of energy turns into heat, and the surface temperature of the ultrasonic probe rises due to the heat generated from the ultrasonic transducers. Because of the generated heat, problems of breakage of the ultrasonic vibrator surface and the acoustic matching layer, deterioration in acoustic characteristics due to separation or the like, degradation in reliability and quality are caused. Further, because of the surface temperature rise of the ultrasonic probe, a problem of low-temperature burn or the like that reduces safety is caused. Accordingly, solving the problems becomes a task.

Here, the three main factors of temperature rise in transmission of ultrasonic waves using an ultrasonic probe are as follows:

(1) The vibration energy of a vibrator itself, which is supplied with a drive signal and expands and contracts, is converted into heat within the vibrator (self-heating);
(2) The ultrasonic waves generated by the vibrator are absorbed by a backing material and converted into heat; and
(3) The ultrasonic waves generated by the vibrator are multiply reflected on the interface of an acoustic matching layer or acoustic lens, and finally converted into heat. The most important factor of them is the factor (1).

Further, with microfabrication of vibrators, increase in the number of vibrators, higher density of vibrators, two-dimensional arrangement of vibrator array, and stacking of vibrator array, the problem of heat generation and surface temperature rise of the ultrasonic probe becomes more serious and it becomes difficult to draw lead wires out from the ultrasonic probe. Accordingly, problems of reduction in reliability, reduction in mass productivity, lower yield, and higher cost are caused. Therefore, solving the problems becomes a task.

As a related technology, Japanese Patent Application Publication JP-P2007-7262A (Document 1) discloses a convex-type ultrasonic probe capable of sufficiently attenuating ultrasonic waves from plural channels of piezoelectric elements toward the backside in a backing member having a convex curved surface, having good heat radiation performance, and capable of relaxing the concentration of heat generation. The ultrasonic probe includes plural channels arranged with desirable spaces in between and each having a piezoelectric element and an acoustic matching layer formed on the piezoelectric element, a support material having a convex curved surface and a coefficient of thermal conductivity of 70 W/(m·K) or more, and a sheet-like acoustic absorbing layer having a homogeneous entire thickness which is glued to the convex curved surface of the support material and on which the piezoelectric elements of the respective channels are mounted and in which grooves are formed in locations corresponding to the spaces of the channels.

Japanese Patent Application Publication JP-P2006-253958A (Document 2) discloses an ultrasonic probe in which plural transducers are arranged in an array form at the leading end. In the ultrasonic probe, the plural ultrasonic transducers are bonded to a flexible sheet having a curved shape, and through holes are perforated in the flexible sheet, in which conducting members electrically connected to the individual electrodes of the plural ultrasonic transducers are embedded.

Japanese Patent Application Publication JP-P2004-363746A (Document 3) discloses an ultrasonic probe including an ultrasonic transducer array in which plural piezoelectric elements provided with electrodes are two-dimensionally arranged, and a wiring material including plural metal wires with sections arranged correspondingly to the arrangement of the plural piezoelectric elements and electrically connected to the electrodes provided in the plural piezoelectric elements, respectively, and a sound absorbing material filling between the plural metal wires.

Japanese Patent Application Publication JP-A-60-68832 (Document 4) discloses an ultrasonic probe including piezoelectric vibrators, a matching layer on the surface at the side of an object to be inspected of the piezoelectric vibrators, and a backing layer at the opposite side to the matching layer of the piezoelectric vibrators. The backing layer is formed by containing metal fibers in a compound material such as synthetic resin or rubber, and the metal fibers are aligned in the same direction as the vibration direction of the piezoelectric vibrators to provide anisotropy in acoustic characteristics.

However, according to Document 1, since the heat generated in the piezoelectric elements transfers to the support material via the acoustic absorbing layer, the heat radiation efficiency is not so much improved unless the coefficient of thermal conductivity of the acoustic absorbing layer is made higher. Documents 2 and 3 disclose secured electric connection to the individual electrodes of the ultrasonic transducer, but do not disclose solution of the problem of temperature rise of the ultrasonic probe due to heat generation. According to Document 4, the backing layer is formed by containing metal fibers in a compound material such as synthetic resin or rubber, and the metal fibers are aligned in the same direction as the vibration direction of the piezoelectric vibrators to provide improved acoustic characteristics. However, it is conceivable that the heat generated in the piezoelectric vibrators transfers to the metal fibers via the compound material, and the radiation efficiency is not so much improved. Further, Document 4 does not disclose improvement in electric connection to the individual electrodes of the piezoelectric vibrators.

Generally, in the case where the piezoelectric vibrators and lead wires are coupled in a one-to-one correspondence, if any one coupling is failed, the entire ultrasonic probe becomes defective and the yield become slower. Additionally, in view of heat radiation, the radiation efficiency is poor because only one lead wire is coupled to one vibrator. Further, heat radiation from the vibrator array is not sufficient because the piezoelectric ceramics such as PZT forming the vibrator is poor in heat conductivity and the epoxy resin, silicone resin, urethane resin, or the like filling between plural vibrators are also poor in heat conductivity. Accordingly, there has been a problem that heat radiation at the central part of the vibrator array becomes especially insufficient and causes a temperature distribution, and the peak temperature becomes higher.

### SUMMARY OF THE INVENTION

The present invention has been achieved in view of the above-mentioned problems. The first purpose of the present invention is to provide a backing material that suppresses the surface temperature rise of an ultrasonic probe. Further, the second purpose of the present invention is to provide a backing material by which lead wires of vibrators can be easily and reliably drawn out. Furthermore, the third purpose of the present invention is to provide an ultrasonic probe, ultrasonic endoscope, ultrasonic diagnostic apparatus, and ultrasonic endoscopic apparatus using such a backing material.

In order to accomplish the purposes, a backing material according to one aspect of the present invention is a backing material provided on a back face of at least one vibrator for transmitting and/or receiving ultrasonic waves in an ultrasonic probe, and includes: a backing base material containing a polymeric material; and a heat conducting fiber provided in the backing base material, having a larger coefficient of thermal conductivity than that of the backing base material, and penetrating the backing base material without disconnection from a first face of the backing material in contact with the at least one vibrator to a second face different from the first face of the backing material.

Further, an ultrasonic probe according to one aspect of the present invention includes: a plurality of vibrators having piezoelectric materials, individual electrodes, and a common electrode, for transmitting and receiving ultrasonic waves; an acoustic matching layer provided in contact with the common electrode of the plural vibrators; and the backing material according to the present invention provided in contact with the individual electrodes of the plural vibrators, the individual electrode of each vibrator coupled to at least two of the heat conducting fibers.

Furthermore, an ultrasonic endoscope according to one aspect of the present invention is an ultrasonic endoscope having an insertion part formed of a material having flexibility and to be used by being inserted into a body cavity of an object to be inspected, and includes in the insertion part: a plurality of vibrators having piezoelectric materials, individual electrodes, and a common electrode, for transmitting and/or receiving ultrasonic waves; an acoustic matching layer provided in contact with the common electrode of the plurality of vibrators; the backing material according to the present invention provided in contact with the individual electrodes of the plural vibrators, the individual electrode of each vibrator coupled to at least two of the heat conducting fibers; illuminating means for illuminating an interior of a body cavity of the object; and imaging means for optically imaging the interior of the body cavity of the object.

In addition, an ultrasonic diagnostic apparatus according to one aspect of the present invention includes: the ultrasonic probe according to the present invention; drive signal supply means for supplying drive signals to the plurality of vibrators; and signal processing means for processing reception signals outputted from the plurality of vibrators to generate image data representing an ultrasonic image. Further, an ultrasonic endoscopic apparatus according to one aspect of the present invention includes: the ultrasonic endoscope according to the present invention; drive signal supply means for supplying drive signals to the plurality of vibrators; and signal processing means for processing reception signals outputted from the plurality of vibrators to generate image data representing an ultrasonic image.

According to the present invention, since the heat conducting fibers having the larger coefficient of thermal conductivity than that of the backing base material, and penetrating the backing base material without disconnection from the first face of the backing material in contact with the at least one vibrator to the second face different from the first face of the backing material are provided, the backing material that suppresses the surface temperature rise of the ultrasonic probe can be provided. Especially, when the individual electrodes of the vibrators are electrically connected to the end surfaces of the plural electrically conducting heat conducting fibers, lead wires of the vibrators can be easily and reliably drawn out.

### BRIEF DESCRIPTION OF THE DRAWINGS

FIG. 1 is a perspective view schematically showing an internal structure of an ultrasonic probe according to the first embodiment of the present invention;

FIG. 2 is a sectional view of the internal structure of the ultrasonic probe shown in FIG. 1 along a plane in parallel with the YZ-plane;

FIG. 3A is a plan view of a backing material according to the first embodiment of the present invention, and FIG. 3B is a perspective view of the backing material according to the first embodiment of the present invention;

FIG. 4 shows measurement results of surface temperature of the ultrasonic probe according to the first embodiment of the present invention in comparison with those in a conventional case;

FIG. 5 is a sectional view showing an internal structure of an ultrasonic probe according to the second embodiment of the present invention;

FIG. 6A is a sectional view showing an internal structure of the ultrasonic probe according to the fourth embodiment of the present invention, and FIG. 6B is a plan view showing an internal structure of the ultrasonic probe according to the fourth embodiment of the present invention;

FIG. 7 shows measurement results of surface temperature of the ultrasonic probe according to the fourth embodiment of the present invention in comparison with those in a conventional case;

FIG. 8 shows structures of piezoelectric vibrator in comparison between the first embodiment and the fifth embodiment of the present invention;

FIG. 9 shows measurement results of surface temperature of an ultrasonic probe according to the fifth embodiment of the present invention in comparison with those in a conventional case;

FIG. 10 shows structures of piezoelectric vibrator in comparison between the fourth embodiment and the sixth embodiment of the present invention;

FIG. 11 is a sectional view showing an internal structure of an ultrasonic probe according to the seventh embodiment of the present invention;

FIG. 12 is a schematic diagram showing an appearance of an ultrasonic endoscope according to one embodiment of the present invention;

FIG. 13A is a plan view showing the upper surface of the leading end of the insertion part shown in FIG. 12, and FIG. 13B is a side sectional view showing the side surface of the leading end of the insertion part shown in FIG. 12; and

FIG. 14 shows an ultrasonic diagnostic apparatus including the ultrasonic probe according to the respective embodiments of the present invention and an ultrasonic diagnostic apparatus main body; and

FIG. 15 shows an ultrasonic endoscopic apparatus including the ultrasonic endoscope according to the one embodiment of the present invention and an ultrasonic endoscopic apparatus main body.

### DESCRIPTION OF THE PREFERRED EMBODIMENTS

Hereinafter, preferred embodiments of the present invention will be explained in detail with reference to the drawings. The same reference numerals will be assigned to the same component elements and the description thereof will be omitted.

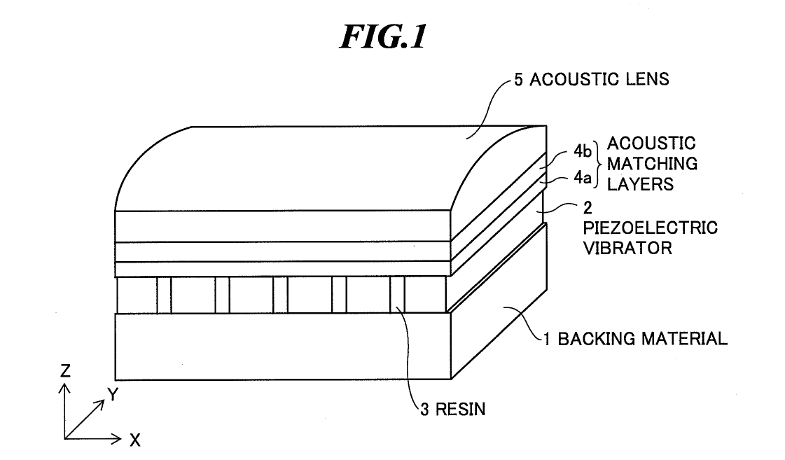

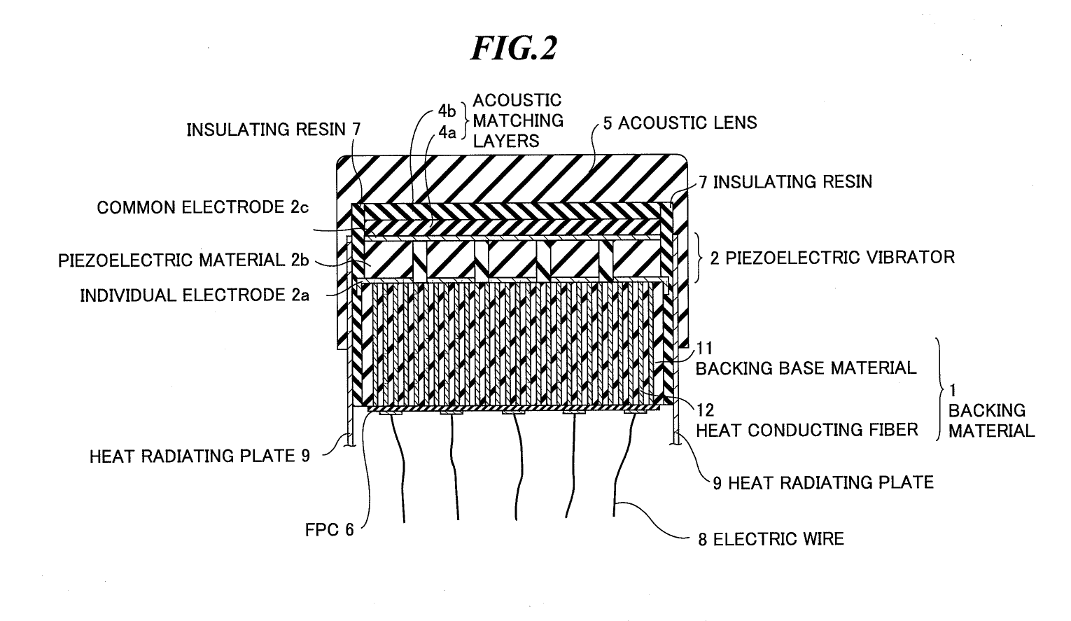

FIG. 1 is a perspective view schematically showing an internal structure of an ultrasonic probe according to the first embodiment of the present invention, and FIG. 2 is a sectional view of the internal structure of the ultrasonic probe shown in FIG. 1 along a plane in parallel with the YZ-plane. The ultrasonic probe is used in contact with an object to be inspected when extracavitary scan is performed or used by being inserted into a body cavity of the object when intracavitary scan is performed.

As shown in FIGS. 1 and 2, the ultrasonic probe has a backing material 1, one or plural ultrasonic transducers (piezoelectric vibrators) 2 provided on the backing material 1, resins 3 provided between those piezoelectric vibrators 2, one or plural acoustic matching layers (two acoustic matching layers 4a and 4b are shown in FIGS. 1 and 2) provided on the piezoelectric vibrators 2, an acoustic lens 5 provided on the acoustic matching layers according to need, two flexible printed circuit boards (FPCs) 6 fixed onto both side surfaces and the bottom surface of the backing material 1, insulating resins 7 formed on the side surfaces of the backing material 1, the piezoelectric vibrators 2, and the acoustic matching layers 4a and 4b via the FPCs 6, and electric wiring 8 connected to the FPCs 6. In FIG. 1, the FPCs 6 to electric wires 8 are omitted and the acoustic lens 5 is partially cut for showing the arrangement of the piezoelectric vibrators 2. In the embodiment, the plural piezoelectric vibrators 2 arranged in the X-axis direction form a one-dimensional vibrator array.

As shown in FIG. 2, each piezoelectric vibrator 2 includes an individual electrode 2a formed on the backing material 1, a piezoelectric material 2b of PZT (Pb(lead) zirconate titanate) or the like formed on the individual electrode 2a, and a common electrode 2c formed on the piezoelectric material 2b. Typically, the common electrode 2c is commonly connected to the ground potential (GND). The individual electrodes 2a of the piezoelectric vibrators 2 are connected to the electric wires 8 via printed wiring formed on the two FPCs 6 fixed onto the both side surfaces and the bottom surface of the backing material 1. The width of the piezoelectric material 2b (in the X-axis direction) is 100 μm, the length (in the Y-axis direction) is 5000 μm, and the thickness (in the Z-axis direction) is 300 μm. The polarization direction of the piezoelectric material 2b is the Z-axis direction.

Further, at least one heat radiating plate (two heat radiating plates 9 are shown in FIG. 2) may be provided on the side surfaces of the backing material 1 and the piezoelectric vibrators 2 via the insulating resins 7. In this case, the heat radiating plate 9 may be connected to a shield layer of a conducting material provided in a cable for connecting the ultrasonic probe to the ultrasonic diagnostic apparatus main body. As a material of the heat radiating plate 9, a metal having a high coefficient of thermal conductivity such as copper (Cu) is used. Further, as the insulating resin 7, a resin having a high coefficient of thermal conductivity is desirably used. The heat generated at the central part of the piezoelectric vibrator 2 moves through the backing material 1 and transfers to the heat radiating plate 9 via the insulating resin 7.

The plural ultrasonic vibrators 2 generate ultrasonic waves based on the drive signals respectively supplied from the ultrasonic diagnostic apparatus main body. Further, the plural ultrasonic vibrators 2 receive ultrasonic echoes propagating from the object and generate plural electric signals, respectively. The electric signals are outputted to the ultrasonic diagnostic apparatus main body and processed as reception signals of the ultrasonic echoes.

The acoustic matching layers 4a and 4b arranged on the front surface of the ultrasonic vibrators 2 are formed of, foe example, Pyrex® glass or an epoxy resin including metal powder, which easily propagates ultrasonic waves, and provides matching of acoustic impedances between the object as a living body and the ultrasonic vibrators 2. Thereby, the ultrasonic waves transmitted from the ultrasonic vibrators 2 efficiently propagate into the object.

The acoustic lens 5 is formed of, for example, silicone rubber, and focuses an ultrasonic beam transmitted from the ultrasonic transducer array 12 and propagating through the acoustic matching layers 4a and 4b at a predetermined depth within the object.

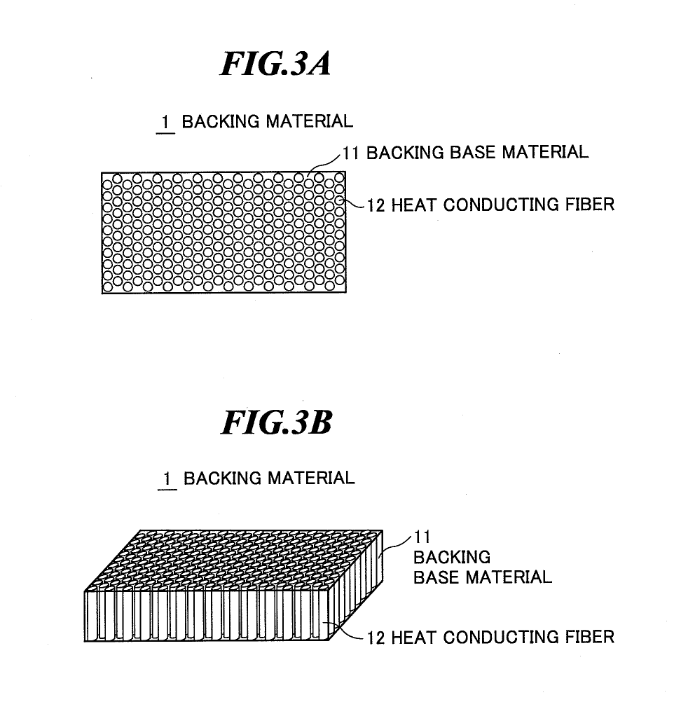

FIG. 3A is a plan view of the backing material in the ultrasonic probe according to the first embodiment of the present invention, and FIG. 3B is a perspective view of the backing material in the ultrasonic probe according to the first embodiment of the present invention. In the first embodiment of the present invention, the backing material 1 includes a backing base material 11 having electric insulation properties, and heat conducting fibers 12 having electric insulation properties provided in the backing base material 11. Here, the heat conducting fibers 12 have higher heat conductivity than that of the backing base material 11.

As shown in FIG. 3B, the heat conducting fibers 12 penetrates the backing base material 11 without disconnection from the first face of the backing material 1 in contact with the plural piezoelectric vibrators (vibrator array) to the second face different from the first face of the backing material 1. The individual electrode 2a of each piezoelectric vibrator 2 provided on the backing material 1 is coupled to at least two heat conducting fibers 12, and improve the radiation efficiency from the piezoelectric vibrators 2. In the case of a convex array probe, the backing material 1 having a shape convex upward is used.

The backing base material 11 is formed of a material having great acoustic attenuation, for example, a polymeric material such as resin or rubber in which ferrite powder, PZT powder, or metal powder is dispersed, and has a role of promoting attenuation of unwanted ultrasonic waves generated from the plural piezoelectric vibrators 2.

In the backing material according to the first embodiment of the present invention, the backing base material 11 is formed by mixing tungsten fine particles in an epoxy resin. For example, the ratio of the epoxy resin is 70 wt %. The tungsten fine particles provide the acoustic attenuation function required for the backing material 1. Further, for electric insulation, the surfaces of the tungsten fine particles are covered by insulating thin films of silicon oxide (SiO₂). The coefficient of thermal conductivity κ of the backing base material 11 is 2 W/m·K.

The heat conducting fibers 12 are formed by applying the same material resin as that of the backing base material 11 to surfaces of aluminum nitride (AlN) fibers and curing it. The diameter of the aluminum nitride fibers is 15 μm, for example, and the diameter of the heat conducting fibers 12 is one size larger than the diameter of the aluminum nitride fibers. The backing material 1 shown in FIGS. 3A and 3B is formed in the following manner, these heat conducting fibers 12 are bundled and the spaces between them are filled with a material to be the backing base material 11 and cured, and then, they are cut and ground. The entire size of the backing material 1 is 50 mm in length (in the X-axis direction), 30 mm in width (in the Y-axis direction), and 10 mm in thickness (in the Z-axis direction), for example.

The heat conducting fibers 12 run through without disconnection from the upper end face to the lower end face of the backing material 1 and exposed on the both end faces. The aluminum nitride (AlN) is an electrically insulating material and has large thermal conductivity with a coefficient of thermal conductivity κ of 200 W/m·K. By adjusting the thickness of the resin coating films applied to the surfaces of the aluminum nitride fibers, the volume fraction of aluminum nitride in the backing material 1 can be adjusted.

If the acoustic impedance of the backing material is matched to the acoustic impedance of the piezoelectric vibrators (e.g., Z=34 Mrayl), the frequency characteristic of the ultrasonic probe covers the wider band, the vibration waveform becomes shorter, and the resolving power in the depth direction becomes higher, while the sensitivity of the ultrasonic probe becomes lower. That is, the frequency characteristic and the sensitivity are in a trade-off relationship. In practice, it is necessary to choice the optimum value according to the purpose in consideration of the trade-off relationship.

In the embodiment, the volume fraction of aluminum nitride is 70%. Under the condition, the acoustic impedance Zb of the backing material in the sound wave traveling direction is 29.2 Mrayl. The following results were obtained by measuring the coefficient of thermal conductivity κ (parallel) in a direction parallel to the sound wave traveling direction and the coefficient of thermal conductivity κ (perpendicular) in a direction perpendicular to the sound wave traveling direction of the backing material according to the embodiment by using the laser flash method.

κ (parallel)=141 W/m·K

κ (perpendicular)=6.5 W/m·K

In comparison with the coefficient of thermal conductivity κ=2 W/m·K of the backing base material 11, both the coefficient of thermal conductivity κ (parallel) in the direction parallel to the sound wave traveling direction and the coefficient of thermal conductivity κ (perpendicular) in the direction perpendicular to the sound wave traveling direction of the backing material according to the embodiment increase. Especially, the coefficient of thermal conductivity κ (parallel) in the direction parallel to the sound wave traveling direction remarkably increased.

For comparison, the measurement result of the coefficient of thermal conductivity of a backing material formed by mixing the aluminum nitride particles at a volume fraction of 70% in the same resin as that of the backing base material 11 was 7 W/m·K. This value is nearly equal to that of the coefficient of thermal conductivity κ (perpendicular) in the direction perpendicular to the sound wave traveling direction of the backing material according to the embodiment.

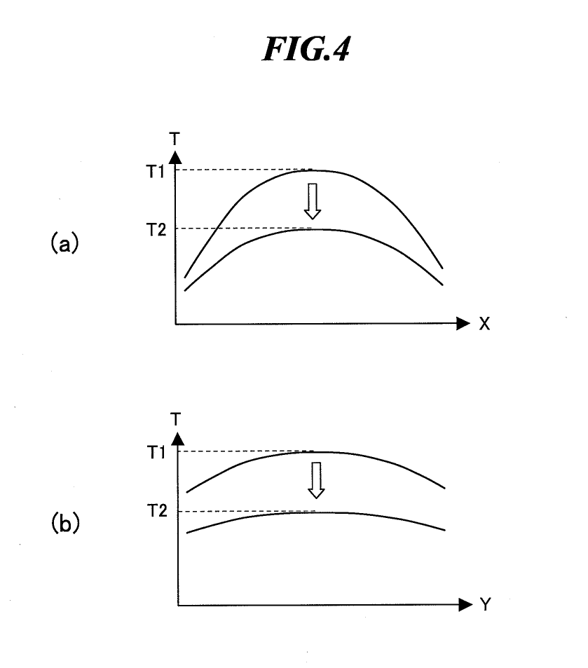

FIG. 4 shows measurement results of surface temperature of the ultrasonic probe according to the first embodiment of the present invention in comparison with those in a conventional case. The measurement was made by measuring the surface temperature of the acoustic lens in the air at a temperature of 23° C. In the ultrasonic probe used in surface temperature measurement, no heat radiating plate 9 shown in FIG. 2 is provided. FIG. 4 (a) shows a temperature distribution in the X-axis direction, which passes through the point of peak temperature on the surface of the acoustic lens, and FIG. 4 (b) shows a temperature distribution in the Y-axis direction, which passes through the point of peak temperature on the surface of the acoustic lens.

The peak temperature T1 on the surface of the acoustic lens of the ultrasonic probe using a conventional backing material containing no aluminum nitride fibers is 39° C., while the peak temperature T2 on the surface of the acoustic lens of the ultrasonic probe using the backing material containing aluminum nitride fibers according to the embodiment is 28° C. Accordingly, the surface temperature of the ultrasonic probe can be reduced by using the backing material containing heat conducting fibers.

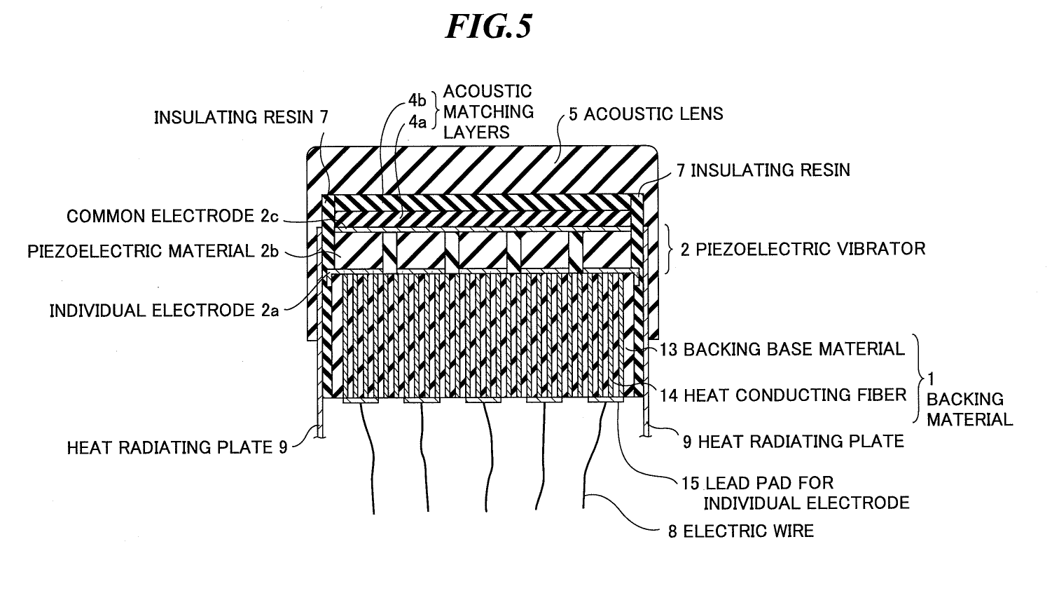

Next, the second embodiment of the present invention will be explained with reference to FIGS. 1 and 5. The second embodiment differs from the first embodiment in that the heat conducting fibers have electric conductivity, and the electric connection between the individual electrodes and electric wires is made by using the heat conducting fibers in this embodiment. The rest of the configuration is the same as that in the first embodiment.

FIG. 5 is a sectional view showing an internal structure of the ultrasonic probe according to the second embodiment of the present invention. As shown in FIG. 5, a piezoelectric vibrator 2 includes an individual electrode 2a formed on the backing material 1, a piezoelectric material 2b of PZT (Pb(lead) zirconate titanate) or the like formed on the individual electrode 2a, and a common electrode 2c formed on the piezoelectric material 2b. Typically, the common electrode 2c is commonly connected to the ground potential (GND).

In the second embodiment, the backing material 1 includes a backing base material 13 having electric insulation properties, and heat conducting fibers 14 having electric conductivity provided in the backing base material 13. Here, the heat conducting fibers 14 have higher heat conductivity than that of the backing base material 13. The individual electrode 2a of each piezoelectric vibrators 2 is electrically connected to two or more heat conducting fibers 14 having electric conductivity, and further electrically connected to an electric wire 8 via a lead pad 15 provided on the bottom of the backing material 1.

In the backing material according to the second embodiment of the present invention, the backing base material 13 is formed by mixing tungsten carbide fine particles in an epoxy-urethane mix rubber. For example, the ratio of the epoxy-urethane mix rubber is 95 wt %. The tungsten carbide fine particles provide the acoustic attenuation function required for the backing material. Further, for electric insulation, the surfaces of the tungsten carbide fine particles are covered by insulating thin films of silicon oxide (SiO₂). The coefficient of thermal conductivity κ of the backing base material 13 is 5 W/m·K.

Here, a method of forming insulating thin films of silicon oxide on the surfaces of tungsten carbide fine particles will be explained. First, using a beaker, a mixture liquid is prepared by adding 20 g of tungsten carbide powder to 200 g of anhydrous alcohol in which alkoxide of silicon has been dissolved. As the alkoxide of silicon, tetra-ethoxy-silane (TEOS:Si(OCH₂CH₃)₄) is used, or tetra-methoxy-silane, tetra-propoxy-silane, tetra-butoxy-silane, or the like may be used.

Then, 100 g of ethanol containing 10 wt % of water in weight concentration is dropped into the beaker of the mixture liquid that has been stirred and suspended so as to decompose (hydrolyze) the alkoxide within the mixture liquid, and thereby, insulating coatings containing silicon oxide (SiO₂) are formed on the surfaces of the tungsten carbide powder. If the weight concentration of the water in the hydrous ethanol to be dropped is high, the ratio of spontaneous nucleation of the alkoxide decomposed matter becomes higher, and formation of coatings by ripening becomes difficult. On the other hand, if the weight concentration of the water in the hydrous ethanol to be dropped is low, the hydrolysis may take a long time, or unreacted matter may remain. Therefore, the weight concentration of the water in the hydrous ethanol to be dropped is preferably set to 50 wt % or less, more preferably set to 5 wt % to 20 wt %.

In order to promote the hydrolysis reaction, it is preferable to heat the mixture liquid during dropping of the hydrous ethanol to a temperature of about 60° C., or to make the mixture liquid mild acidic of about pH2 to pH4 by adding hydrochloric acid or the like to the hydrous ethanol to be dropped. Further, in order to complete the hydrolysis reaction, it is preferable that destructive distillation may be performed for several hours while heating is kept after dropping of the hydrous ethanol. Then, the mixture liquid is held at a temperature of 100° C. for evaporation of the liquid component, and further, the dried powder is held at a temperature of 300° C. for two hours. Thereby, dense coatings can be formed.

Further, the heat conducting fibers 14 are formed by electro-depositing an insulating resin on the surfaces of cupper fibers for electric insulation, and applying the same material resin as that of the backing base material 13 thereon and curing it. The diameter of the copper fibers is 20 μm, for example, and the diameter of the heat conducting fibers 14 is one size larger than the diameter of the copper fibers. The backing material 1 as shown in FIGS. 3A and 3B is formed in the following manner, these heat conducting fibers 14 are bundled and the spaces between them are filled with a material to be the backing base material 13 and cured, and then, they are cut and ground.

The coefficient of thermal conductivity κ of the copper fibers is 390 W/m·K. When the volume fraction of the copper in the backing material was set to 70%, the coefficient of thermal conductivity κ (parallel) in a direction parallel to the sound wave traveling direction (in a direction in which the heat conducting fibers are oriented) of the backing material according to the embodiment was 275 W/m·K, the coefficient of thermal conductivity κ (perpendicular) in a direction perpendicular to the sound wave traveling direction was 16 W/m·K, and the acoustic impedance Z in the direction parallel to the sound wave traveling direction was 31.1 Mrayl.

Next, the third embodiment of the present invention will be explained. In an ultrasonic probe according to the third embodiment of the present invention, carbon (C) fibers having electric conductivity are used as the heat conducting fibers of the backing material. The backing base material is fabricated by mixing tungsten fine particles at 70 wt % in an epoxy resin as is the case of the first embodiment. The coefficient of thermal conductivity κ of the backing base material in the embodiment is 2 W/m·K.

The diameter of the carbon fibers is 10 μm, for example. The coefficient of thermal conductivity κ in the longitudinal direction of the carbon fibers is 1000 W/m·K. When the volume fraction of the copper in the backing material was set to 50%, the coefficient of thermal conductivity κ (parallel) in a direction in which the heat conducting fibers of the backing material are oriented was 501 W/m·K, the coefficient of thermal conductivity κ (perpendicular) in a direction perpendicular thereto was 3.8 W/m·K, and the acoustic impedance Z in the direction parallel to the sound wave traveling direction was 31.9 Mrayl.

Next, the fourth embodiment of the present invention will be explained. In an ultrasonic probe according to the fourth embodiment of the present invention, a two-dimensional vibrator array is provided on a backing material in which heat conducting fibers are provided through therein. In the fourth embodiment, for example, the backing material according to any one of the first to third embodiments may be used, and the case of using the backing material according to the third embodiment will be explained as below.

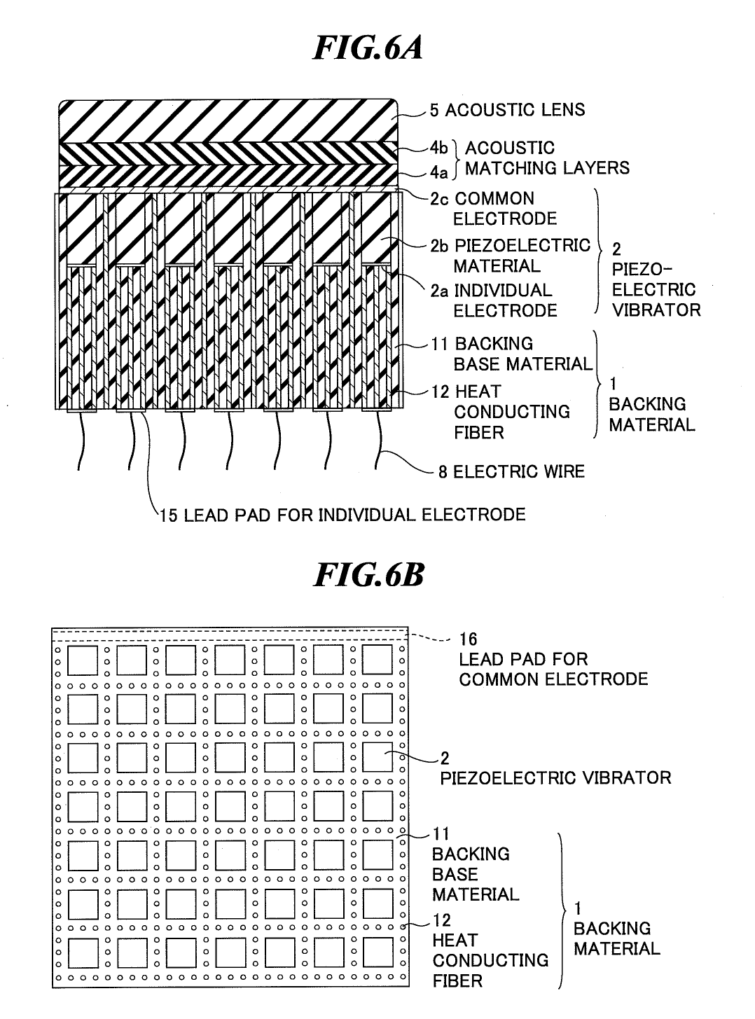

FIG. 6A is a sectional view showing an internal structure of the ultrasonic probe according to the fourth embodiment of the present invention, and FIG. 6B is a plan view showing an internal structure of the ultrasonic probe according to the fourth embodiment of the present invention. In FIG. 6B, the upper layers than the common electrode are omitted. In the embodiment, the two-dimensional vibrator array is formed by embedding and arranging the plural piezoelectric vibrators 2 into the backing material 1.

As shown in FIGS. 6A and 6B, each piezoelectric vibrator 2 includes an individual electrode 2a formed on the backing material 1, a piezoelectric material 2b formed on the individual electrode 2a, and a common electrode 2c formed on the piezoelectric material 2b. The individual electrode 2a and the common electrode 2c are formed by sputtering, for example. The size of individual piezoelectric vibrators 2 is 300 μm×300 μm×600 μm, for example. On the two-dimensional vibrator array, one or plural acoustic matching layers (two acoustic matching layers 4a and 4b are shown in FIGS. 6A and 6B) and an acoustic lens 5 (according to need) are formed.

Plural carbon fibers as the heat conducting fibers 12 are coupled to the individual electrode 2a of each piezoelectric vibrator 2. The individual electrode 2a of each piezoelectric vibrator 2 is electrically connected to the plural carbon fibers and further electrically connected to an electric wire 8 via a lead pad 15 (lead pad for individual electrode) provided on the lower face of the backing material 1. The common electrode 2c is also electrically connected to an electric wire 8 via a lead pad 16 (lead pad for common electrode) provided on the lower face of the backing material 1. Since the plural carbon fibers are connected to each piezoelectric vibrator 2, the probability of defective connection can be reduced and the reliability can be improved. Further, the manufacturing yield is improved and the cost is reduced.

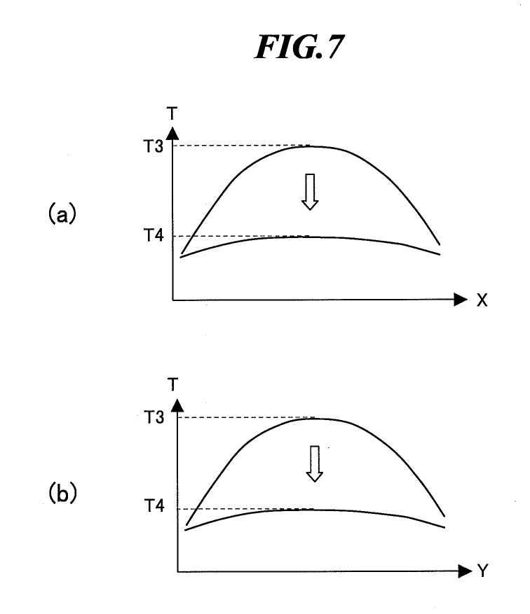

FIG. 7 shows measurement results of surface temperature of the ultrasonic probe according to the fourth embodiment of the present invention in comparison with those in a conventional case. The measurement was made by measuring the surface temperature of the acoustic lens in the air at a temperature of 23° C. FIG. 7 (a) shows a temperature distribution in the X-axis direction, which passes through the point of peak temperature on the surface of the acoustic lens, and FIG. 7 (b) shows a temperature distribution in the Y-axis direction, which passes through the point of peak temperature on the surface of the acoustic lens.

The peak temperature T3 on the surface of the acoustic lens of the ultrasonic probe using a conventional backing material containing no heat conducting fibers is 43° C., while the peak temperature T4 on the surface of the acoustic lens of the ultrasonic probe using the backing material containing heat conducting fibers according to the embodiment is 26° C. Accordingly, it is known that the surface temperature of the ultrasonic probe can be reduced by using the backing material containing heat conducting fibers.

Next, the fifth embodiment of the present invention will be explained. An ultrasonic probe according to the fifth embodiment uses multilayered piezoelectric vibrators in the one-dimensional vibrator array of the ultrasonic probe according to the first to third embodiments of the present invention. As below, the case using the multilayered piezoelectric vibrators in the ultrasonic probe according to the first embodiment will be explained.

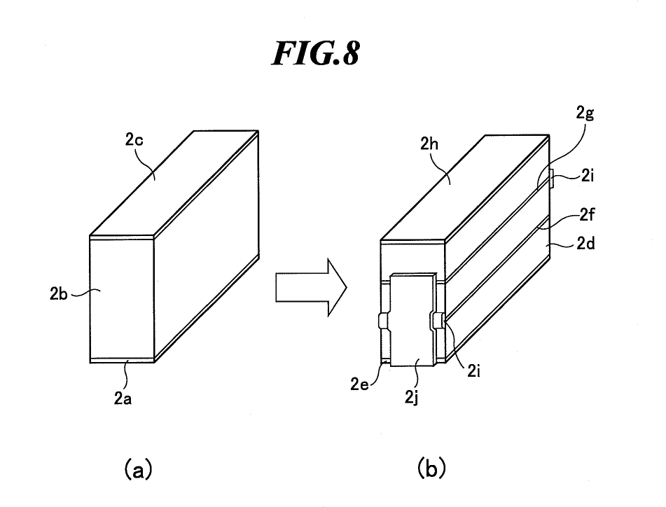

FIG. 8 shows structures of piezoelectric vibrator in comparison between the first embodiment and the fifth embodiment of the present invention. In the first embodiment, as shown in FIG. 8 (a), the piezoelectric vibrator includes the individual electrode 2a, the piezoelectric material 2b formed on the individual electrode 2a, and the common electrode 2c formed on the piezoelectric material 2b, and has a single-layered structure.

On the other hand, in the fifth embodiment, as shown in FIG. 8 (b), a piezoelectric vibrator includes plural piezoelectric material layers 2d formed of PZT or the like, a lower electrode layer 2e, internal electrode layers 2f and 2g alternately inserted between the plural piezoelectric material layers 2d, an upper electrode layer 2h, insulating films 2i, and a front side electrode 2j an a rear side electrode 2k (not shown), and has a multilayered structure.

Here, the lower electrode layer 2e is connected to the front side electrode 2j and insulated from the rear side electrode. The upper electrode layer 2h is connected to the rear side electrode and insulated from the front side electrode 2j. Further, the internal electrode layer 2f is connected to the rear side electrode and insulated from the front side electrode 2j by the insulating film 2i. On the other hand, the internal electrode layer 2g is connected to the front side electrode 2j and insulated from the rear side electrode by the insulating film 2i. The electrodes of an ultrasonic transducer are formed in this fashion, three pairs of electrodes for applying electric fields to the three layers of piezoelectric vibrator layers 2d are connected in parallel. The number of piezoelectric vibrator layers is not limited to three, but may be two or four or more.

In the multilayered piezoelectric vibrator, the area of opposed electrodes becomes larger than that of the single-layered element, and the electric impedance becomes lower. Therefore, the multilayered piezoelectric vibrator operates more efficiently for the applied voltage than the single-layered piezoelectric vibrator having the same size. Specifically, given that the number of piezoelectric material layers is N, the number of the multilayered piezoelectric vibrator is N-times the number of piezoelectric material layers of the single-layered piezoelectric vibrator and the thickness of each layer of the multilayered piezoelectric vibrator is 1/N of the thickness of each layer of the single-layered piezoelectric vibrator, and the electric impedance of the multilayered piezoelectric vibrator is 1/N²-times the electric impedance of the single-layered piezoelectric vibrator. Therefore, the electric impedance of piezoelectric vibrator can be adjusted by increasing or decreasing the number of stacked piezoelectric material layers, and thus, the electric impedance matching between a drive circuit or signal cable and itself is easily provided, and the sensitivity can be improved. On the other hand, the capacitance is increased due to the stacked form of the piezoelectric vibrator, the amount of heat generated in each piezoelectric vibrator becomes larger. The increased amount of heat is transferred to the outside of the ultrasonic probe by using the backing material into which the heat conducting fibers are inserted, and the temperature rise of the ultrasonic probe is prevented.

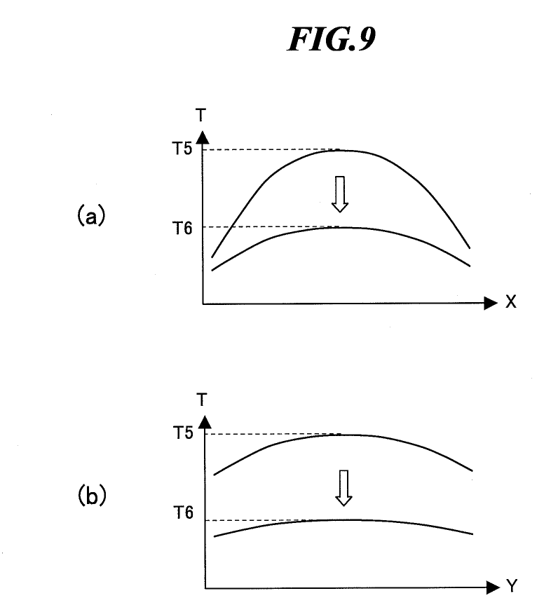

FIG. 9 shows measurement results of surface temperature of the ultrasonic probe according to the fifth embodiment of the present invention in comparison with those in a conventional case. The measurement was made by measuring the surface temperature of the acoustic lens in the air at a temperature of 23° C. In the ultrasonic probe used in surface temperature measurement, no heat radiating plate 9 shown in FIG. 2 is provided. FIG. 9 (a) shows a temperature distribution in the X-axis direction, which passes through the point of peak temperature on the surface of the acoustic lens, and FIG. 9 (b) shows a temperature distribution in the Y-axis direction, which passes through the point of peak temperature on the surface of the acoustic lens.

Since the ultrasonic probe has the multilayered structure, the amount of generated heat is larger and the peak temperature is higher than those of the first embodiment of the present invention. The peak temperature T5 on the surface of the acoustic lens of the ultrasonic probe using a conventional backing material containing no heat conducting fibers is 77° C., while the peak temperature T6 on the surface of the acoustic lens of the ultrasonic probe using the backing material containing heat conducting fibers according to the embodiment is 35° C. Accordingly, it is known that the surface temperature of the ultrasonic probe can be reduced by using the backing material containing heat conducting fibers.

The sixth embodiment of the present invention will be explained. The ultrasonic probe according to the sixth embodiment uses multilayered piezoelectric vibrators in the two-dimensional vibrator array of the ultrasonic probe according to the fourth embodiment.

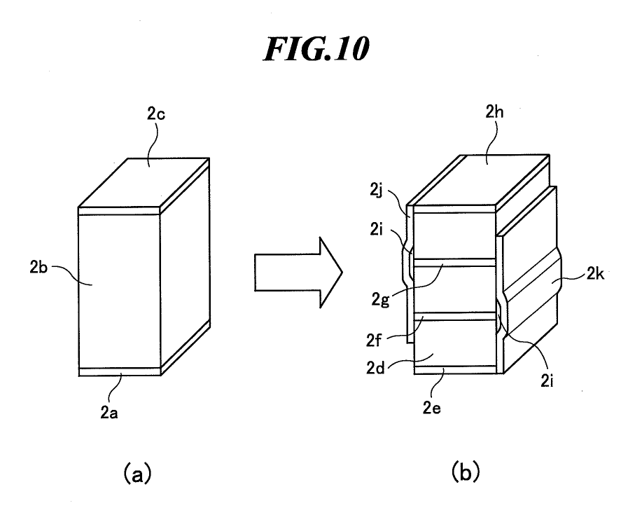

FIG. 10 shows structures of piezoelectric vibrator in comparison between the fourth embodiment and the sixth embodiment of the present invention. In the fourth embodiment shown in FIG. 10 (a), the piezoelectric vibrator includes the individual electrode 2a, the piezoelectric material 2b formed on the individual electrode 2a, and the common electrode 2c formed on the piezoelectric material 2b, and has a single-layered structure.

On the other hand, in the sixth embodiment shown in FIG. 10 (b), a piezoelectric vibrator includes plural piezoelectric material layers 2d formed of PZT or the like, a lower electrode layer 2e, internal electrode layers 2f and 2g alternately inserted between the plural piezoelectric material layers 2d, an upper electrode layer 2h, insulating films 2i, and side electrodes 2j and 2k, and has a multilayered structure.

Here, the lower electrode layer 2e is connected to the side electrode 2k at the right side in the drawing and insulated from the side electrode 2j at the left side in the drawing. The upper electrode layer 2h is connected to the side electrode 2j and insulated from the side electrode 2k. Further, the internal electrode layer 2f is connected to the side electrode 2j and insulated from the side electrode 2k by the insulating film 2i. On the other hand, the internal electrode layer 2g is connected to the side electrode 2k and insulated from the side electrode 2j by the insulating film 2i. The electrodes of an ultrasonic transducer are formed in this fashion, three pairs of electrodes for applying electric fields to the three layers of piezoelectric vibrator layers 2d are connected in parallel. The number of piezoelectric vibrator layers is not limited to three, but may be two or four or more.

The capacitance is increased due to the stacked form of the piezoelectric vibrator, the amount of heat generated in each piezoelectric vibrator becomes larger. The increased amount of heat is transferred to the outside of the ultrasonic probe using the backing material into which the heat conducting fibers are inserted, and thereby, the temperature rise of the ultrasonic probe can be prevented.

In the sixth embodiment of the present invention, measurement results of surface temperature of the acoustic lens in the air at a temperature of 23° C. will be explained. In the sixth embodiment, since the ultrasonic vibrator has the multilayered structure, the amount of generated heat is larger and the peak temperature on the surface of the acoustic lens 5 is higher than those of the fourth embodiment. The peak temperature T7 on the surface of the acoustic lens of the ultrasonic probe using a conventional backing material containing no carbon fibers is 79° C., while the peak temperature T8 on the surface of the acoustic lens of the ultrasonic probe using the backing material containing carbon fibers according to the embodiment is 33° C. Accordingly, it is known that the surface temperature of the ultrasonic probe can be reduced by using the backing material containing heat conducting fibers.

The seventh embodiment of the present invention will be explained. The ultrasonic probe according to the seventh embodiment uses a backing material in which electrically insulating heat conducting fibers and electrically conducting heat conducting fibers are provided through and a one-dimensional or two-dimensional vibrator array.

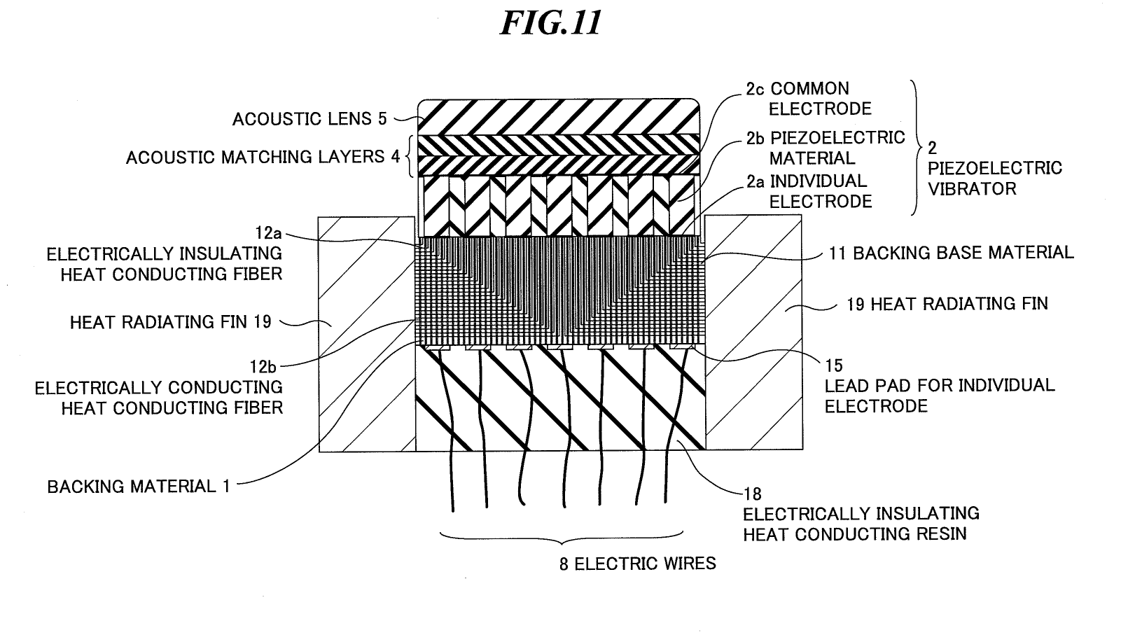

FIG. 11 is a sectional view showing an internal structure of the ultrasonic probe according to the seventh embodiment of the present invention. As shown in FIG. 11, inside the backing material 1, electrically insulating heat conducting fibers 12a are folded toward the side faces in the backing material 1, and thermally coupled to piezoelectric vibrators 2 and thermally coupled to heat radiating fins 19 provided on the side faces of the backing material 1. On the other hand, electrically conducting heat conducting fibers 12b are inserted from the upper end face to the lower end face of the backing material 1, and electrically connected to the piezoelectric vibrators 2, electrically connected to the lead pads 15, and further, electrically connected to electric wires 8 via the lead pads 15.

The electrically insulating heat conducting fibers 12a are formed of aluminum nitride (AlN) fibers having a diameter of 10 μm, for example. The electrically conducting heat conducting fibers 12b are formed of carbon fibers having a diameter of 15 μm, for example. The backing base material 11 is formed by mixing tungsten (W) fine particles in an epoxy resin as is the case of the first embodiment. The radiation property may be further improved by filling the bottom part of the backing material 1 with an electrically insulating heat conducting resin 18.

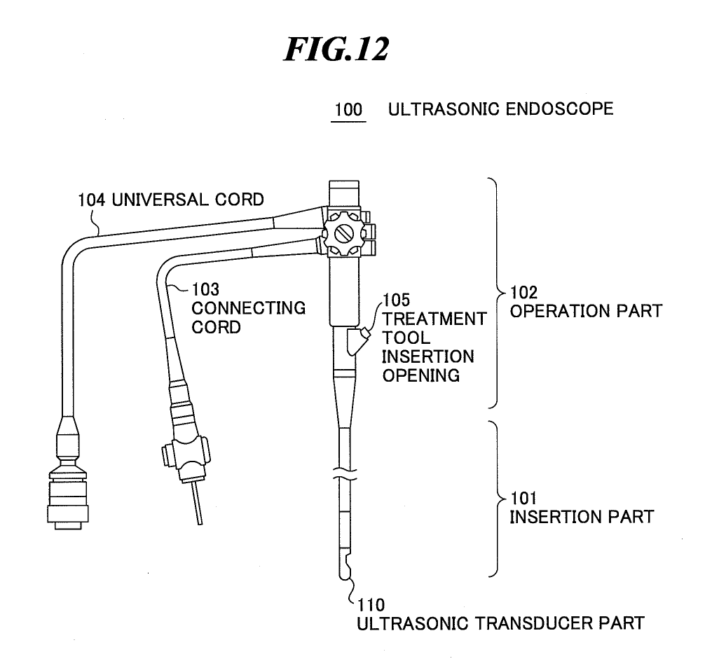

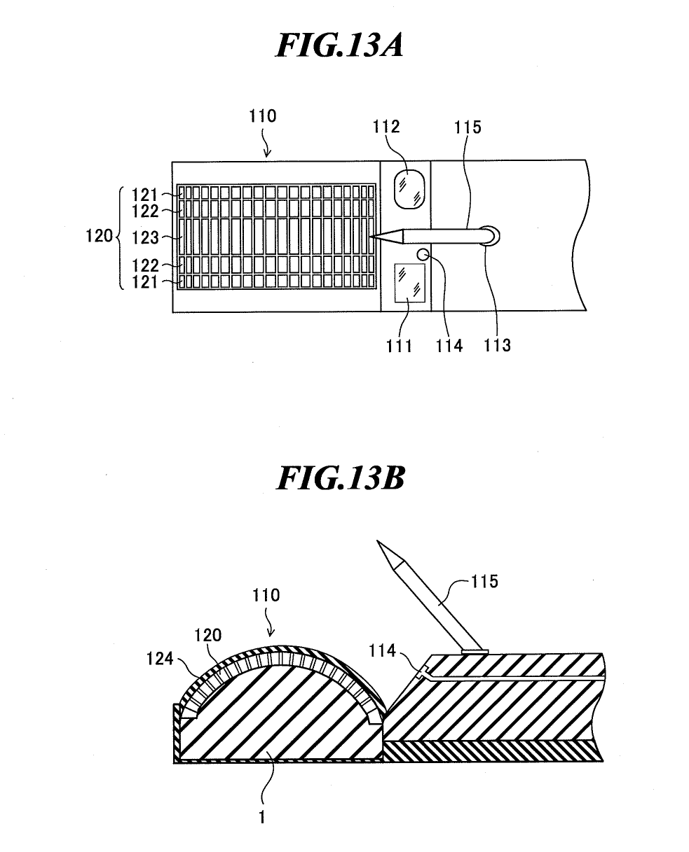

Next, an ultrasonic endoscope according to one embodiment of the present invention will be explained with reference to FIGS. 12 and 13. The ultrasonic endoscope refers to an apparatus provided with an ultrasonic transducer part at the leading end of an insertion part of an endoscopic examination unit for optically observing the interior of the body cavity of the object.

FIG. 12 is a schematic diagram showing an appearance of the ultrasonic endoscope according to the one embodiment of the present invention. As shown in FIG. 12, an ultrasonic endoscope 100 includes an insertion part 101, an operation part 102, a connecting cord 103, and a universal cord 104. The insertion part 101 of the ultrasonic endoscope 100 is an elongated tube formed of a material having flexibility for insertion into the body of the object. An ultrasonic transducer part 110 is provided at the leading end of the insertion part 101. The operation part 102 is provided at the base end of the insertion part 101, connected to the ultrasonic endoscopic apparatus main body via the connecting cord 103, and connected to a light source unit via the universal cord 104. A treatment tool insertion opening 105 for inserting a treatment tool or the like into the insertion part 101 is provided in the operation part 102.

FIG. 13A is a plan view showing the upper surface of the leading end of the insertion part shown in FIG. 12, and FIG. 13B is a side sectional view showing the side surface of the leading end of the insertion part shown in FIG. 12. In FIG. 13A, an acoustic matching layer 124 shown in FIG. 13B is omitted.

As shown in FIGS. 13A and 13B, at the leading end of the insertion part, the ultrasonic transducer part 110, an observation window 111, an illumination window 112, a treatment tool passage opening 113, and a nozzle hole 114 are provided. A punctuation needle 115 is provided in the treatment tool passage opening 113. In FIG. 13A, an objective lens is fit in the observation window 111, and an input end of an image guide or a solid-state image sensor such as a CCD camera is provided in the imaging position of the objective lens. These configure an observation optics. The observation optics may be configured such that the image entering from the observation window 111 via the objective lens is taken out of the ultrasonic endoscope via the image guide and imaging or observation can be performed at the output end of the image guide. Further, an illumination lens for outputting illumination light to be supplied from the light source unit via a light guide is fit in the illumination window 112. These configure an illumination optics.

The treatment tool passage opening 113 is a hole for leading out a treatment tool or the like inserted from the treatment tool insertion opening 105 provided in the operation part 102 shown in FIG. 12. Various treatments are performed within a body cavity of the object by projecting the treatment tool such as the punctuation needle 115 or forceps from the hole and operating it with the operation part 102. The nozzle hole 114 is provided for injecting a liquid (water or the like) for cleaning the observation window 111 and the illumination window 112.

The ultrasonic transducer part 110 includes a convex-type multirow array, and a vibrator array 120 has plural ultrasonic transducers (piezoelectric vibrators) 121-123 arranged in five rows on a curved surface. As shown in FIG. 13B, the acoustic matching layer 124 is provided on the front side of the vibrator array 120. An acoustic lens is provided on the acoustic matching layer 124 according to need. Further, a backing material 125 is provided on the back side of the vibrator array 120.

In FIGS. 13A and 13B, the convex-type multirow array is shown as the vibrator array 120, however, a radial-type ultrasonic transducer part in which plural ultrasonic transducers are arranged on a cylindrical surface, or an ultrasonic transducer part in which plural ultrasonic transducers are arranged on a spherical surface may be used. In the embodiment, in the ultrasonic transducer part, the backing material as that of the ultrasonic probe according to any one of the first to seventh embodiments of the present invention, the radiation structure and/or the electric connection structure using the backing material are used.

As above, the respective embodiments of the present invention have been explained, however, the present invention is not limited to those. For example, plural kinds of heat conducting fibers having different diameters may be used in combination as heat conducting fibers to be provided in the backing base material. Further, although the examples using aluminum nitride (AlN) as the material of the electrically insulating heat conducting fibers have been described, other materials such as aluminum oxide (Al₂O₃), silicon oxide (SiO₂), diamond (C), or boron nitride (BN) may be used. Although the examples using copper (Cu) or carbon fibers as the material of the electrically conducting heat conducting fibers have been described, a metal such as gold (Au), silver (Ag), or aluminum (Al), or a metal compound such as silicon carbide (SiC) or tungsten carbide (WC) may be used. In view of the coefficient of thermal conductivity, carbon fibers or metal is desirable.

Although the examples using tungsten (W) and tungsten carbide (WC) as fine particles to be mixed into the backing base material have been described, other materials such as tungsten boride (WB), tungsten nitride (WN), and ferrite may be used. Further, in order to improve the coefficient of thermal conductivity, particles such as diamond particles, black lead particles, metal particles, silicon carbide (SiC), aluminum nitride (AlN), tungsten carbide (WC), boron nitride (BN), or aluminum oxide (Al₂O₃) may be mixed.

The backing materials according to the first to seventh embodiments of the present invention have been explained by taking an example of a backing material having a planar shape, however, a backing material having a curved shape such as a convex shape may be used. The backing material shown in FIGS. 13A and 13B has been explained by taking an example of a backing material having a convex shape, however, a backing material having a planar shape may be used.

In the ultrasonic probe and ultrasonic endoscope according to the respective embodiments, an acoustic matching layer having a lower coefficient of thermal conductivity and/or an acoustic lens having a lower coefficient of thermal conductivity may be used for reducing the surface temperature. Further, in the ultrasonic probe and ultrasonic endoscope according to the respective embodiments, the heat may be released from the backing material to a casing or cable.

Furthermore, in the ultrasonic probe according to the fourth or sixth embodiments of the present invention, the ultrasonic probe is formed as a two-dimensional array probe such that the plural vibrators of the two-dimensional array may be individually driven, however, the ultrasonic probe may be formed as a single composite piezoelectric element driven as a single element by forming an overall electrode.

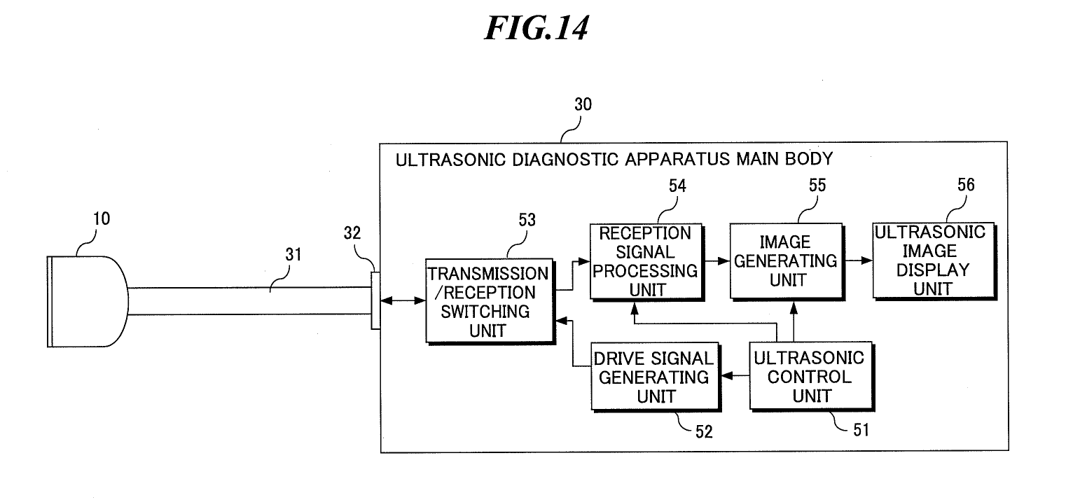

FIG. 14 shows an ultrasonic diagnostic apparatus including the ultrasonic probe according to the respective embodiments of the present invention and an ultrasonic diagnostic apparatus main body. As shown in FIG. 14, the ultrasonic probe 10 is electrically connected to the ultrasonic diagnostic apparatus main body 30 via an electric cable 31 and an electric connector 32. The electric cable 31 transmits drive signals generated in the ultrasonic diagnostic apparatus main body 30 to the respective ultrasonic transducers and transmits reception signals outputted from the respective ultrasonic transducers to the ultrasonic diagnostic apparatus main body 30.

The ultrasonic diagnostic apparatus main body 30 includes an ultrasonic control unit 51 for controlling the imaging operation using the ultrasonic transducers, a drive signal generating unit 52, a transmission/reception switching unit 53, a reception signal processing unit 54, an image generating unit 55, and an ultrasonic image display unit 56. The drive signal generating unit 52 includes plural drive circuits (pulsers or the like), for example, and generates drive signals to be used for respectively driving the plural ultrasonic transducers. The transmission/reception switching unit 53 switches output of drive signals to the ultrasonic probe 10 and input of reception signals from the ultrasonic probe 10.

The reception signal processing unit 54 includes plural preamplifiers, plural A/D converters, and a digital signal processing circuit or CPU, for example, and performs predetermined signal processing of amplification, phase matching and addition, detection, or the like on the reception signals outputted from the ultrasonic transducers. The image generating unit 55 generates image data representing an ultrasonic image based on the reception signals on which the predetermined signal processing has been performed. The ultrasonic image display unit 56 displays the ultrasonic image based on thus generated image data.

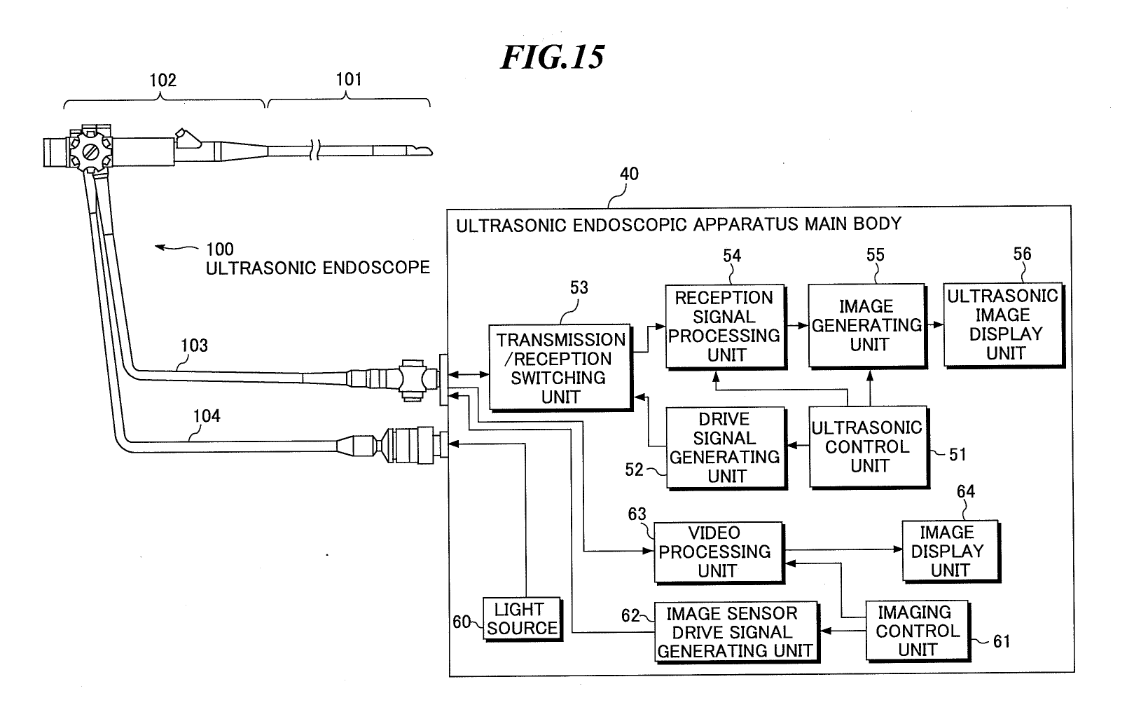

FIG. 15 shows an ultrasonic endoscopic apparatus including the ultrasonic endoscope and the ultrasonic endoscopic apparatus main body according to the one embodiment of the present invention. The plural ultrasonic transducers are electrically connected to the ultrasonic endoscopic apparatus main body 40 via the insertion part 101, the operation part 102, and the connecting cord 103. Plural shield lines transmit drive signals generated in the ultrasonic endoscopic apparatus main body 40 to the respective ultrasonic transducers and transmit reception signals outputted from the respective ultrasonic transducers to the ultrasonic endoscopic apparatus main body 40.

The ultrasonic endoscopic apparatus main body 40 includes the ultrasonic control unit 51, the drive signal generating unit 52, the transmission/reception switching unit 53, the reception signal processing unit 54, the image generating unit 55, the ultrasonic image display unit 56, a light source 60, an imaging control unit 61, an image sensor drive signal generating unit 62, a video processing unit 63, and an image display unit 64.

The ultrasonic control unit 51, the drive signal generating unit 52, the transmission/reception switching unit 53, the reception signal processing unit 54, the image generating unit 55, and the ultrasonic image display unit 56 have been already explained with respect to the ultrasonic wave diagnostic apparatus and the description thereof will be omitted. The light source 60 emits light to be used for illumination of the object. The light outputted from the light source 60 illuminates the object via the universal cord 104 through the illumination window 112 of the insertion part 101 (FIG. 13A). The illuminated object is imaged by the image sensor through the observation window 111 of the insertion part 101 (FIG. 13A), and video signals outputted from the image sensor are inputted to the video processing unit 63 of the ultrasonic endoscopic apparatus main body 40 via the connecting cord 103.

The imaging control unit 61 controls imaging operation using the image sensor. The image sensor drive signal generating unit 62 generates drive signals to be supplied to the image sensor. The video processing unit 63 generates image data based on the video signals to be inputted from the image sensor. The image display unit 64 inputs the image data from the video processing unit 63 and displays images of the object.

1. A backing material provided on a back face of at least one vibrator for transmitting and/or receiving ultrasonic waves in an ultrasonic probe, said backing material comprising:
a backing base material containing a polymeric material; and
a heat conducting fiber provided in said backing base material, having a larger coefficient of thermal conductivity than that of said backing base material, and penetrating said backing base material without disconnection from a first face of said backing material in contact with said at least one vibrator to a second face different from the first face of said backing material.

2. The backing material according to claim 1, wherein said backing base material further contains inorganic fine particles dispersed in said polymeric material, said inorganic fine particles containing at least one of a metal material, tungsten carbide (WC), tungsten boride (WB), tungsten nitride (WN), ferrite, diamond, black lead, silicon carbide (SiC), aluminum nitride (AlN), boron nitride (BN), and aluminum oxide (Al₂O₃), said metal material including tungsten (W).

3. The backing material according to claim 1, wherein said heat conducting fiber includes an electrically insulating heat conducting fiber formed of an electrically insulating material.

4. The backing material according to claim 1, wherein said heat conducting fiber includes an electrically conducting heat conducting fiber formed of an electrically conducting material.

5. The backing material according to claim 4, wherein said heat conducting fiber further includes an electrically insulating coating formed around said electrically conducting heat conducting fiber.

6. The backing material according to claim 1, wherein said heat conducting fiber includes an electrically insulating heat conducting fiber formed of an electrically insulating material and an electrically conducting heat conducting fiber formed of an electrically conducting material.

7. The backing material according to claim 6, wherein said heat conducting fiber further includes an electrically insulating coating formed around said electrically conducting heat conducting fiber.

8. An ultrasonic probe comprising:
a plurality of vibrators having piezoelectric materials, individual electrodes, and a common electrode, for transmitting and/or receiving ultrasonic waves;
an acoustic matching layer provided in contact with the common electrode of said plurality of vibrators; and
a backing material provided in contact with the individual electrodes of said plurality of vibrators, said backing material including a backing base material containing a polymeric material, and heat conducting fibers provided in said backing base material, having a larger coefficient of thermal conductivity than that of said backing base material, and penetrating said backing base material without disconnection from a first face of said backing material in contact with said plurality of vibrators to a second face different from the first face of said backing material, the individual electrode of each vibrator coupled to at least two of said heat conducting fibers.

9. The ultrasonic probe according to claim 8, wherein said heat conducting fibers include electrically conducting heat conducting fibers formed of an electrically conducting material, and the individual electrode of each vibrator is electrically connected to end surfaces of at least two of said electrically conducting heat conducting fibers.

10. The ultrasonic probe according to claim 9, wherein said plurality of vibrators are embedded in said backing material, and the common electrode of said plurality of vibrators is electrically connected to end surfaces of at least two of said electrically conducting heat conducting fibers.

11. The ultrasonic probe according to claim 9, further comprising:
a plurality of lead pads provided on the second face of said backing material and electrically connected to said individual electrodes and/or said common electrode.

12. An ultrasonic endoscope having an insertion part formed of a material having flexibility and to be used by being inserted into a body cavity of an object to be inspected, said ultrasonic endoscope comprising in said insertion part:
a plurality of vibrators having piezoelectric materials, individual electrodes, and a common electrode, for transmitting and/or receiving ultrasonic waves;
an acoustic matching layer provided in contact with the common electrode of said plurality of vibrators;
a backing material provided in contact with the individual electrodes of said plurality of vibrators, said backing material including a backing base material containing a polymeric material, and heat conducting fibers provided in said backing base material, having a larger coefficient of thermal conductivity than that of said backing base material, and penetrating said backing base material without disconnection from a first face of said backing material in contact with said plurality of vibrators to a second face different from the first face of said backing material, the individual electrode of each vibrator coupled to at least two of said heat conducting fibers;
illuminating means for illuminating an interior of a body cavity of the object; and
imaging means for optically imaging the interior of the body cavity of the object.

13. An ultrasonic diagnostic apparatus comprising:
an ultrasonic probe including a plurality of vibrators having piezoelectric materials, individual electrodes, and a common electrode, for transmitting and/or receiving ultrasonic waves, an acoustic matching layer provided in contact with the common electrode of said plurality of vibrators, and a backing material provided in contact with the individual electrodes of said plurality of vibrators, said backing material including a backing base material containing a polymeric material, and heat conducting fibers provided in said backing base material, having a larger coefficient of thermal conductivity than that of said backing base material, and penetrating said backing base material without disconnection from a first face of said backing material in contact with said plurality of vibrators to a second face different from the first face of said backing material, the individual electrode of each vibrator coupled to at least two of said heat conducting fibers;
drive signal supply means for supplying drive signals to said plurality of vibrators; and
signal processing means for processing reception signals outputted from said plurality of vibrators to generate image data representing an ultrasonic image.

14. An ultrasonic endoscopic apparatus comprising:
an ultrasonic endoscope having an insertion part formed of a material having flexibility and to be used by being inserted into a body cavity of an object to be inspected, said ultrasonic endoscope including in said insertion part, a plurality of vibrators having piezoelectric materials, individual electrodes, and a common electrode, for transmitting and/or receiving ultrasonic waves, an acoustic matching layer provided in contact with the common electrode of said plurality of vibrators, a backing material provided in contact with the individual electrodes of said plurality of vibrators, said backing material including a backing base material containing a polymeric material, and heat conducting fibers provided in said backing base material, having a larger coefficient of thermal conductivity than that of said backing base material, and penetrating said backing base material without disconnection from a first face of said backing material in contact with said plurality of vibrators to a second face different from the first face of said backing material, the individual electrode of each vibrator coupled to at least two of said heat conducting fibers, illuminating means for illuminating an interior of a body cavity of the object, and imaging means for optically imaging the interior of the body cavity of the object;
drive signal supply means for supplying drive signals to said plurality of vibrators; and
signal processing means for processing reception signals outputted from said plurality of vibrators to generate image data representing an ultrasonic image.
